(19) United States
(12) Patent Application Publication
(10) Pub. No.: US 2013/0345629 A1
(43) Pub. Date: Dec. 26, 2013

(54) COATING AND COATING METHOD FOR THE BALLOON OF A BALLOON CATHETER, AND BALLOON CATHETER WITH COATED BALLOON

(71) Applicant: Alexander Rübben, Saint Leon (MC)

(72) Inventor: Alexander Rübben, Saint Leon (MC)

(21) Appl. No.: 13/991,343

(22) PCT Filed: Dec. 5, 2011
(86) PCT No.: PCT/DE2011/075300

§ 371 Date: Aug. 12, 2013

(30) Foreign Application Priority Data
Dec. 4, 2010 (DE) 20 2010 016 123.1
Jan. 26, 2011 (DE) 10 2011 000 340.1
Jun. 14, 2011 (DE) 10 2011 051 059.1

(51) Int. Cl. A61L 29/08; A61L 29/16

(52) U.S. Cl. CPC A61L 29/085; A61L 29/16
USPC 604/103.02; 427/2.3

(57) ABSTRACT

The invention relates to an active-substance-containing coating for the balloon of a balloon catheter, to a balloon of a balloon catheter with the coating, and to a balloon catheter with such a balloon. The invention further relates to the use of the coating for a balloon of a balloon catheter, and to a method for coating an active-substance-coated balloon and for producing an agent for modifying the release of an active substance applied to a surface of the balloon of a balloon catheter. A coating for a balloon of a balloon catheter is proposed which comprises at least one lipophilic active substance and an agent for modifying the release of active substance to a vessel that surrounds the balloon, wherein the agent for modifying the release of active substance is a polysaccharide having a molecular mass of 10,000 Da to 100,000,000 Da.

### TECHNICAL FIELD OF THE INVENTION

The invention relates to a coating for the balloon of a balloon catheter, to a balloon of a balloon catheter with the coating, and to a balloon catheter with such a balloon. The invention further relates to the use of the coating for a balloon of a balloon catheter, and to a method for coating an active-substance-coated balloon and for producing an agent for modifying the release of an active substance applied to a surface of the balloon of a balloon catheter.

### BACKGROUND OF THE INVENTION

The so-called “minimal invasive techniques” in medicine offer possibilities to cardiologist and radiologists for treatment or diagnosis of vessels, but may bear a risk of vascular wall thickening with a consecutive lumen constriction due to cell proliferation. To counteract such a risk, active-substance-containing coatings for release of drugs from the surface of a balloon of a balloon catheter are known in prior art.

WO 2008/061842 A2 discloses methods in which the surface of a balloon of a balloon catheter is structured and/or profiled chemically or by roughening in order to improve an active-substance-containing coating on a balloon.

WO 2009/124570 A1 discloses a method in which an active substance is embedded into the surface of the balloon by softening the surface of a balloon of a balloon catheter.

WO 2010/009904 A2 discloses a method in which an active-substance-containing coating is embrittled on the surface of a balloon of a balloon catheter.

Solubilizers, e.g. phosphatidylcholine, polyethoxylated castor oil or a mixture of cardiolipine, phosphatidycholine, and cholesterol by means of which active substances can be trapped into micelles that are outwardly hydrophilic due to solubilizers, are known for handling of lipophilic active substances, e.g. paclitaxel, WO 2010/136604 A1 discloses active-substance micelles and, as an alternative, active-substance particles suspended by means of tenside solubilizers and embedded into polymer coatings, WO 00/32267A2 and US 2002/0123505 A1 disclose polymer coatings as active-substance carriers.

For handling of lipophilic active-substances on embedding into the surface of a balloon of a balloon catheter, WO 2004/028610 A2 teaches a low-molecular matrix substance composed of sugars having a molecular weight of less than 5,000 Da, and WO 2000/121840 discloses a shellac coating with paclitaxel. EP 1689092 A1 discloses a colloidal magnetite coated with degraded dextran to serve as carrier for an antihyperplastic active agent

### SUMMARY OF THE INVENTION

It is the object of the present invention to provide modified active-substance-containing coatings with lipophilic active substances of the balloons of balloon catheters, more particularly with a retarded and/or long-term active substance release upon inflation and pressing a balloon against the vascular tissue surrounding the balloon, more particularly with less expenditure on reproducibility, quality assurance and/or manufacture. Preferably if is intended to enable a release of the active substance in such a manner that 48 hours after the dilatation of the balloon there still are active-substance concentrations in the vascular tissue which has surrounded the balloon on dilatation that still correspond to ½ to 1/10, preferably to ⅓ to ⅕ of the active-substance concentrations that existed two hours after dilatation of the balloon in the vascular tissue surrounding the balloon on dilatation. Furthermore it shall preferably be made possible to deliver the active substance in the long term in such a manner that several days after dilatation of the balloon there still will exist significant active-substance concentrations in that vascular tissue that has surrounded the balloon during dilatation. The present invention also intends to specify an accordingly improved balloon for a balloon catheter, a balloon catheter with an accordingly improved balloon as well as a method for producing an agent for modifying the release of an active substance applied on a surface of a balloon of a balloon catheter by way of which if is possible to exert a positive influence on the release of an active substance with regard to the a.m. retarded and/or long-term release.

Under another aspect, it is intended to enable a release of the active substance in such a manner that active-substance concentrations still exist in the vascular tissue some weeks upon dilatation of the balloon.

Under one aspect of the present invention, the modified active-substance coating enables a favourable pharmacokinetics m accordance with which long-term increased and effective active-substance concentrations exist in the tissue that has surrounded a balloon on its expansion.

The present invention also provides for measures to ensure that the agent for modifying is well mixed with the actual active substance and/or has an especially fine distribution.

In accordance with such measures, one aspect of the present invention is providing a method for generating a bioactive surface on the balloon of a balloon catheter, and another aspect is providing a coating and the products contained therein to form the bioactive surface.

The afore-mentioned object is achieved by a solubilizer-free coating for a balloon of a balloon catheter which at least comprises a lipophilic active substance and an agent for modifying the release of the active substance to a vessel surrounding the balloon, wherein the agent for modifying the release of the active substance is a polysaccharide (polymeric carbohydrate) with a mean molecular mass of 10,000 Da to 100,000 Da. more particularly 20,000 Da to 500,000 Da. The polysaccharide is preferably a branched polysaccharide A solubilizer-free coating is a coating which is produced without addition of solubilizers.

The polysaccharide may be a natural polysaccharide or a modified polysaccharide or a mixture of various polysaccharides and/or modified polysaccharides. Preferably used is a branched and/or modified polysaccharide Particularly eligible for use as modified polysaccharide is hydroxyethyl starch (HES).

Preferably applied as polysaccharide is a natural dextran. In general, natural dextrans are industrially produced enzymatically by bacteria or yeasts from saccharose, and they have mean molecular masses ranging between 10,000 and 50,000,000 Da

In a preferred embodiment of the inventive method in which dextran is utilized as polysaccharide, the dextran has a molecular mass ranging between 20,000 and 80,000 Da. Preferably used is Dextran 40, a dextran having a mean molecular mass of approximately 40,000 Da.

It is supposed that after a vascular dilatation by a balloon provided with an inventive coating, more particularly with an inventive dextran-containing coating, a polysaccharide and/or dextran matrix with the active substance adheres to the vascular wall even after removal of the balloon and that a delivery of the active substance from this matrix is realized to the tissue of the vessel, with the delivery being realized by this matrix in a retarded and/or long-term manner. It is especially the use of dextran having a mean molecular mass of 20,000 Da to 80,000 Da, advantageously 40,000 Da to 80,000 Da and particularly preferably the use of Dextran 40 that enables a long-term release of the active substance. By means of animal experiments on a pig it was possible to verify that it was possible to achieve a long-term release of the active substance by applying the present invention in such a manner that 48 hours upon dilatation of the balloon in the vascular tissue which surrounded the balloon on dilatation, there still existed active-substance concentrations that corresponded to ½ to 1/10, more particularly to ⅓ to ⅕ of the active-substance concentration which existed two hours upon dilatation of the balloon.

An inventive coating can be produced by at least partly moistening the surface of a lipophilic active-substance-coated balloon of a balloon catheter with a solution. This solution is comprised of at least one polysaccharide and at least one solvent. When the solvent is removed from the moistened balloon, a coating according to the present invention remains on the balloon.

With one inventive coating for a balloon of a balloon catheter having a layer of a lipophilic active substance and at least one agent for modifying the release of the active substance to a vessel surrounding the balloon, wherein the agent for modifying the release of the active substance is a polysaccharide having a mean molecular mass of 10,000 Da to 100,000,000 Da. the agent for modifying the release of the active substance is available in form of a layer which is directly or indirectly provided on the active-substance layer side averted from the balloon which also achieves the afore-mentioned object. This coating may feature all the aspects of the solubilizer-free coating as mentioned before.

With an inventive balloon of a balloon catheter, the lipophilic active substance may advantageously be arranged directly on the surface of the balloon, and the polysaccharide may cover the active substance and the balloon. Thus, the balloon can easily be produced by use of conventional standard balloons with the active-substance coating in particular by
moistening the active-substance-coated surface of the balloon at least partly, preferably completely with a solution comprising at least one afore-mentioned polysaccharide and at least one solvent, and by
separating the solvent from the polysaccharide.

For applying the active substance, the surface of the balloon, more particularly a polymeric surface, can be moistened at least partly with a first solution of an active substance, more particularly of paclitaxel in an organic solvent, for example a conventional paclitaxel methylenchloride solution.

Subsequently, at least the part of the balloon surface moistened with the first solution of an active substance can inventively be moistened with a second solution comprised of the afore-mentioned polysaccharide, preferably a dextran solution, in which a water-alcohol mixture serves as solvent. This solution preferably has a contents of polysaccharide of 1-15% by weight, further preferably 2 to 10% by weight, further preferably 3-8% by weight, more particularly 5% by weight, wherein a preferred polysaccharide is Dextran 40. Furthermore, the water-alcohol mixture features an alcohol content of 25-50% by weight, further preferably 30 to 45% by weight, further preferably 35 to 42% by weight, more particularly 39% by weight, wherein the alcohol contents is preferably formed by ethanol.

The balloon surface moistened with the first solution of an active substance is preferably dried quickly, for example within 2 to 7 minutes.

By moistening the surface, of the balloon with the first solution of a lipophilic active substance and by drying, a lacquer-like transparent active-substance layer is generated on the surface. This moistening has proved to be the basis for a homogeneous and reproducible active-substance loading and it advantageously serves for applying the entire active-substance loading.

By moistening with the second solution, preferably an aqueous dextran-alcohol solution, the entire coating becomes more brittle and optically less transparent, i.e. more milky. The surface thus generated has a chalk-like consistency. It is supposed that with the inventive coating formation in the second solution it is the alcohol that enables embrittlement of the active substance and that dextran molecules, in particular, can sediment between the molecules of the active substance, more particularly paclitaxel, in order to achieve a homogeneously fine distribution of the active substance in the polysaccharide-active substance coating and thereby a particularly long release of the active substance to the surrounding tissue in a human body.

The long-term release, preferably over 1 to 3 weeks, more particularly 1 to 2 weeks upon dilatation of a balloon of a balloon catheter provided with an inventive active-substance coating, results in a delivery of effective dosages of the active substance to the tissue which surrounded the balloon on dilatation.

In principle, both the entire balloon surface and only part of the balloon surface, for example the area of the surface which gets in contact with the tissue when expanded, can be coated by applying the inventive method.

The invention also relates to a balloon catheter with an inventive balloon as well as to the use of the inventive coating for a balloon of a balloon catheter.

Balloons which can be provided with an inventive coating and/or which can he subjected to an inventive method are disclosed, e.g. in the printed publications WO 2008/061642 A2, WO 2009/124570 A1, and WO 2010/009904 A2.

According to the present invention, an agent for modifying the release of a lipophilic active substance applied on the surface of a balloon of a balloon catheter can be formed by
moistening the surface of a balloon of a balloon catheter at least partly with a solution containing at least one afore-mentioned polysaccharide and at least one solvent, and by
separating the solvent from the polysaccharide.

The polysaccharide may be a natural polysaccharide such as preferably Dextran, more particularly Dextran with a mean molecular mass ranging between 20,000 and 80,000 Da, advantageously 40,000-80,000 Da, or a modified polysaccharide such as hydroxyethyl starch (HES), more particularly HES with a mean molecular mass ranging between 100,000 and 400,000 Da, or one can use a mixture comprised of various of these carbohydrates.

Accordingly, the active substance may form a structure with deepenings on the surface of the balloon, the moistening with the solution and the separation of solvent and polysaccharide being effected in such a manner that the polysaccharide penetrates at least partly into the deepenings.

The solvent can be separated from the polysaccharide by drying.

The solvent may be water or an aqueous solvent mixture. Such a solvent mixture preferably contains a light-volatile organic constituent, particularly an alcohol, particularly ethanol. Drying can be accelerated with an organic constituent such as ethanol as compared with pure water serving as solvent. The organic constituent advantageously is an alcohol a ketone or another organic compound having a higher steam pressure than water and being mixable with water. Examples are methanol, ethanol, isopropanol and aceton. A polar protic solvent constituent such as preferably water is advantageous for the solution.

The solution with which the balloon is moistened may be a saturated solution.

Moistening of the balloon is preferably effected by submerging the at least partly inflated balloon into the solution.

A separation of the solvent by drying can preferably be effected by letting it volatize at air. Measures like heat input or generating a negative pressure are not necessary to form an advantageous coating.

Upon removal of the solvent, the balloon can be folded or rolled-up.

Usable as active substance with the present invention are, for example, tretinoine and/or tretinoine derivates and/or orphanreceptoragonists and/or elafine derivates and/or corticosteroides and/or steroide hormones and/or paclitaxel and/or sirolism and/or paclitaxel derivates and/or raparmune and/or tacrolimus and/or proteins and/or peptides, and/or cell-proliferation-changing substances. Usable as steroide hormones are in particular methylprednisolon, dexamethason or oestradiol. Advantageously used as active substance are substances generally changing cell proliferation.

### DESCRIPTION OF PREFERRED EMBODIMENTS

### EXAMPLE 1

A dextran solution with 5% by wt, Dextran 40 was produced, the solvent being a water-ethanol mixture with 39% by wt. ethanol.

A balloon type ELUTAX 16350 from Aachen Resonance GmbH, D-52074 Aachen, sized 3-5 mm by diameter and 16 mm by length provided with a paclitaxel layer having a loading concentration of 2 micrograms/mm² was used. The balloon was submerged into the dextran solution and pulled out at a velocity of 1 cm/second over its length of 16 mm.

When the balloon had been pulled out entirely from the solution, the balloon catheter was dried at air.

### EXAMPLE 2

In an animal experiment on an anaesthetized pig, a dilatation with a balloon type ELUTAX 16350 and one dilatation with a balloon obtained according to the a.m. example 1 were effected on one animal in two comparable vessels each. The animal was killed after two days, whereupon those areas of the vessels subjected to dilatations were removed and briefly flushed in order to determine the concentration of paclitaxel per mg of the removed vessel samples 48 hours alter the dilatations, 48 hours after the relevant dilatation, the vessel area dilated with the balloon of the afore-mentioned example 1 evidenced a more than 100-fold higher paclitaxel concentration in the tissue as compared with the vessel area dilated with the balloon type ELUTAX 16350.

This result shows the effect of the dextran layer for the long-term and/or retarded release of the active substance paclitaxel.

1-15. (canceled)

16. Balloon for a balloon catheter, comprising a coating, wherein the coating comprises
a layer of a lipophilic active substance, and
at least one agent for modifying the release of active substance to a vessel surrounding the balloon,
wherein the agent for modifying the release of active substance is a polysaccharide with a mean molecular mass of 10,000 Da to 100,000 Da and the agent for modifying the release of active substance is available in form of a layer applied on the side of the layer of the lipophilic active substance that is averted from the balloon.

17. Balloon according to claim 16, wherein the polysaccharide is a natural polysaccharide or a modified polysaccharide or a mixture of various of these polysaccharides.

18. Balloon according to claim 17, wherein the polysaccharide is a natural dextran.

19. Balloon according to claim 18, wherein the natural dextran has a mean molecular mass ranging between 20,000 and 80,000 Da.

20. Balloon according to claim 16, wherein the active substance is arranged on a surface of the balloon and the polysaccharide covers the active substance.

21. Balloon catheter, comprising a balloon according to any of the preceding claims 16 to 20.

22. Method for coating a balloon of a balloon catheter, said balloon being coated on a surface with a lipophilic active substance, wherein
the active-substance-coated surface of the balloon is moistened at least partly with a solution containing at least one polysaccharide having a mean molecular mass of 10,000 Da to 100,000 Da and at least one solvent, and
the solvent is separated from the polysaccharide.

23. Method according to claim 22, wherein the balloon is moistened completely with the solution containing at least one polysaccharide.

24. Method according to claim 22, wherein the active substance forms a structure with deepenings on the surface of the balloon and the separation of the solvent and polysaccharide is realized in such a manner that the polysaccharide penetrates at least partly into the deepening.

25. Method according to claim 22, wherein the polysaccharide is a natural polysaccharide or a modified polysaccharide or a mixture of these polysaccharides.

26. Method according to claim 25, wherein the polysaccharide is a natural dextran.

27. Method according to claim 26, wherein the natural dextran has a mean molecular mass ranging between 20,000 and 80,000 Da.

28. Method according to claim 22, wherein the solvent is separated from the polysaccharide by drying.

29. Method according to claim 22, wherein the solvent is an aqueous solvent mixture.
